(19) United States
(12) Patent Application Publication
(10) Pub. No.: US 2026/0315413 A1
(43) Pub. Date: Oct. 15, 2026

(54) METHODS OF TREATING CHEMOTHERAPY INDUCED PERIPHERAL NEUROPATHY

(71) Applicant: The Trustees of Indiana University, Bloomington, IN (US)

(72) Inventors: Fletcher A. White, Indianapolis, IN (US); Tyler Nguyen, Indianapolis, IN (US)

(21) Appl. No.: 19/465,363

(22) Filed: Jan. 30, 2026

Related U.S. Application Data
(60) Provisional application No. 63/752,272, filed on Jan. 31, 2025.

(51) Int. Cl. A61K 31/165; A61K 31/277; A61K 31/282; A61K 31/64; A61K 33/243; A61P 25/02

(52) U.S. Cl. CPC A61K 31/165; A61K 31/277; A61K 31/282; A61K 31/64; A61K 33/243; A61P 25/02

(57) ABSTRACT

Disclosed are methods of reducing or inhibiting the development of chemotherapy induced peripheral neuropathy, the methods comprising administering a tetrodotoxin-dependent voltage gated sodium channel (NaV) inhibitor and a platinum-based chemotherapy to the subject. The NaV inhibitor may comprise lacosamide. Also disclosed are pharmaceutical compositions and kits comprising NaV inhibitors and platinum-based chemotherapies.

### CROSS-REFERENCE TO RELATED APPLICATIONS

This application claims the benefit of U.S. Appl. No. 63/752,272 filed on Jan. 31, 2025, the content of which is incorporated herein by reference in its entirety.

### STATEMENT REGARDING FEDERALLY SPONSORED RESEARCH OR DEVELOPMENT

This invention was made with government support under NS102415 awarded by National Institutes of Health. The Government has certain rights in the invention.

### BACKGROUND

Oxaliplatin-associated Chemotherapy-induced Peripheral Neuropathy (CIPN) is a frequent, potentially severe and dose-limiting toxicity of colorectal cancer treatment. The overall incidence of CIPN is estimated to be approximately upwards of 40% in patients. CIPN can persist for months to years beyond chemotherapy completion, causing significant challenges for cancer survivors due to its negative influence on quality of life (QOL). CIPN represents an important challenge because of the lack of treatment that can effectively prevent or mitigate this adverse drug effect. Accordingly, there is a need in the art for new therapies for CIPN.

### SUMMARY

In an aspect of the current disclosure, methods are provided. In some embodiments, the methods comprise administering a tetrodotoxin-dependent voltage gated sodium channel (NaV) inhibitor and a platinum-based chemotherapy to a subject. In some embodiments, the methods are methods of reducing or inhibiting the development of chemotherapy induced peripheral neuropathy (CIPN) in a subject in need thereof and the methods comprise administering a tetrodotoxin-dependent voltage gated sodium channel (NaV) inhibitor and a platinum-based chemotherapy to the subject. In some embodiments, the tetrodotoxin-dependent voltage gated sodium channel (NaV) inhibitor is administered prior to the platinum-based chemotherapy, the tetrodotoxin-dependent voltage gated sodium channel (NaV) inhibitor is administered at about the same time as the platinum-based chemotherapy, or the tetrodotoxin-dependent voltage gated sodium channel (NaV) inhibitor is administered after the platinum-based chemotherapy. In some embodiments, the NaV inhibitor is administered at about the same time as the platinum-based chemotherapy. In some embodiments, the NaV inhibitor is lacosamide. In some embodiments, the platinum-based chemotherapy comprises oxaliplatin, carboplatin, cisplatin, or nedaplatin. In some embodiments, the platinum-based chemotherapy comprises oxaliplatin. In some embodiments, the subject is a mammal. In some embodiments, the subject is a human, a dog, a cat, a horse, a mouse, a rat, or a non-human primate. In some embodiments, the subject is a human. In some embodiments, the method reduces mechanical allodynia in the subject. In some embodiments, the subject is being administered a platinum-based chemotherapy for the treatment of a cell proliferative disease or disorder. In some embodiments, the cell proliferative disease or disorder comprises cancer. In some embodiments, the cancer comprises colorectal cancer, testicular cancer, ovarian cancer, lung cancer, bladder cancer, cervical cancer, breast cancer, head and neck cancer, endometrial cancer, prostate cancer, leukemia, sarcoma, stomach cancer, pancreatic cancer, esophageal cancer, uterine cancer, or brain cancer. In some embodiments, the cancer comprises colorectal cancer. In some embodiments, the cancer comprises breast cancer.

In an aspect of the current disclosure, methods of treating chemotherapy induced peripheral neuropathy (CIPN) in a subject in need thereof are provided and comprise administering a tetrodotoxin-dependent voltage gated sodium channel (NaV) inhibitor to a subject that has been administered a platinum-based chemotherapy. In some embodiments, the NaV inhibitor is lacosamide. In some embodiments, the platinum-based chemotherapy comprises oxaliplatin, carboplatin, cisplatin, or nedaplatin. In some embodiments, the platinum-based chemotherapy comprises oxaliplatin. In some embodiments, the subject is a mammal. In some embodiments, the subject is a human, a dog, a cat, a horse, a mouse, a rat, or a non-human primate. In some embodiments, the subject is a human. In some embodiments, the method reduces mechanical allodynia in the subject. In some embodiments, the subject is being administered a platinum-based chemotherapy for the treatment of a cell proliferative disease or disorder. In some embodiments, the cell proliferative disease or disorder comprises cancer. In some embodiments, the cancer comprises colorectal cancer, testicular cancer, ovarian cancer, lung cancer, bladder cancer, cervical cancer, breast cancer, head and neck cancer, endometrial cancer, prostate cancer, leukemia, sarcoma, stomach cancer, pancreatic cancer, esophageal cancer, uterine cancer, or brain cancer. In some embodiments, the cancer comprises colorectal cancer. In some embodiments, the cancer comprises breast cancer.

In an aspect of the current disclosure, methods of treating chemotherapy induced peripheral neuropathy (CIPN) in a subject in need thereof are provided. In some embodiments, the methods comprise administering a NOD, LRR, and pyrin domain-containing protein 3 (NLRP3) inhibitor to a subject that has been administered a platinum-based chemotherapy. In some embodiments, the NLRP3 inhibitor is administered prior to the platinum-based chemotherapy, the NLRP3 inhibitor is administered at about the same time as the platinum-based chemotherapy, or the NLRP3 inhibitor is administered after the platinum-based chemotherapy. In some embodiments, the NLRP3 inhibitor is administered at about the same time as the platinum-based chemotherapy. In some embodiments, the NLRP3 inhibitor is MCC950. In some embodiments, the platinum-based chemotherapy comprises oxaliplatin, carboplatin, cisplatin, or nedaplatin. In some embodiments, the platinum-based chemotherapy comprises oxaliplatin. In some embodiments, the subject is a mammal. In some embodiments, the subject is a human, a dog, a cat, a horse, a mouse, a rat, or a non-human primate. In some embodiments, the subject is a human. In some embodiments, the method reducing mechanical allodynia in the subject. In some embodiments, the subject is or has received a platinum-based chemotherapy for the treatment of a cell proliferative disease or disorder. In some embodiments, the cell proliferative disease or disorder comprises cancer. In some embodiments, the cancer comprises colorectal cancer, testicular cancer, ovarian cancer, lung cancer, bladder cancer, cervical cancer, breast cancer, head and neck cancer, endometrial cancer, prostate cancer, leukemia, sarcoma, stomach cancer, pancreatic cancer, esophageal cancer, uterine cancer, or brain cancer. In some embodiments, the cancer comprises colorectal cancer. In some embodiments, the cancer comprises breast cancer. In some embodiments, method further comprising administering an antineoplastic therapy to the subject in addition to the platinum-based chemotherapy. In some embodiments, the antineoplastic therapy comprises a chemotherapy, a radiation therapy, a hormone therapy, a targeted therapy, or an immunotherapy.

In an aspect of the current disclosure, pharmaceutical compositions are provided. In some embodiments, the pharmaceutical compositions comprise a platinum-based chemotherapy and a tetrodotoxin-dependent voltage gated sodium channel (NaV) inhibitor. In some embodiments, the NaV inhibitor comprises lacosamide. In some embodiments, the platinum-based chemotherapy comprises oxaliplatin, carboplatin, cisplatin, or nedaplatin. In some embodiments, the platinum-based chemotherapy comprises oxaliplatin.

In an aspect of the current disclosure, kits are provided. In some embodiments, the kits comprise a platinum-based chemotherapy and a tetrodotoxin-dependent voltage gated sodium channel (NaV) inhibitor. In some embodiments, the NaV inhibitor comprises lacosamide. In some embodiments, the platinum-based chemotherapy comprises oxaliplatin, carboplatin, cisplatin, or nedaplatin. In some embodiments, the platinum-based chemotherapy comprises oxaliplatin.

### BRIEF DESCRIPTION OF THE FIGURES

FIG. 1 shows that oxaliplatin induced robust caspase-1 mediated inflammatory response at 24 hours after treatment. Co-treatment of Oxaliplatin+MCC950 (NLRP3 specific inhibitor) prevent oxaliplatin-induced inflammatory signals. Dorsal root ganglion (DRG) was harvested and cultured. Oxaliplatin was treated at 10 mins, 3 hours, or 24 hours prior to imaging. Oxaplitin+MCC950 were co-treated at 24 hours prior to imaging (n=5 animals per group; 4 wells of 6-well rectangular plate per animals, ****p<0.0001, one way ANOVA, Tukey's HSD)

FIGS. 2A and 2B shows cranial window preparation in a mouse. The mouse skull was thinned and strengthened by high-strength transparent surgical cyanoacrylate glue (blue area). Sample image shows the thinned-skull window position of the transparent cranial window (B). B, the thinned cranial hemisphere becomes transparent (right) compared to the not-yet-thinned side (left).

FIGS. 3A and 3B show that oxaliplatin treatment produced prolonged increase in caspase-1 activation reporter bioluminescence intensity in multi-body areas up to at least one week after the last injection, in vivo. A, Animal was exposed to an oxaliplatin intraperitoneal injection every two days for a total of 10 days. (total of 5 treatments). Sample images show caspase-1 activation reporter mouse bioluminescence signals at baseline, 24 hours, 3 days, 1 week, and 2 weeks after the injection. B &C Bioluminescent signals associated with caspase-1 activation were significantly elevated in both hindpaws and brain and hindpaws as early as 1 day following last oxaliplatin injection and sustained for at least 3 days. (n=5 mice per group; *p<0.05, **p<0.01, ***p<0.001, ****p<0.0001, repeated-ANOVA, Tukey's HSD).

FIGS. 4A, and 4B, shows that oxaliplatin treatment increases caspase-1 activation reporter bioluminescence intensity in the hindpaw (A) and brain (B) were eliminated when the NLRP3 inhibitor, MCC950, was co-administered with oxaliplatin (n=5 mice per group; *p<0.05, **p<0.01, ***p<0.001, ****p<0.0001, repeated-ANOVA, Tukey's HSD)

FIG. 5 shows that MCC950 treatments attenuated Oxaliplatin (Oxa)-induced mechanical allodynia. Mice were treated with either Oxa or Oxa+MCC950 intraperitoneally at 10 mg/kg (5 mg/mL) every two days for 10 days (total 5 treatments). Greater hypersensitivity of hind paw withdrawal to tactile stimulus in oxaliplatin-treated group, in both male and female animals at 24 hours and lasted for at least 2-weeks post-oxaliplatin treatment. Significant attenuation of mechanical pain hypersensitivity is apparent in animals subjected to the combination of Oxa+MCC950 in both male and female mice. A statistically significant increase in response thresholds (i.e., less pain) was detected in the Oxa+MCC950-treated group at 24 hours and 1 week after last treatment compared to the oxa-treated group. (n=5 mice per sex per group; *p<0.05, two-way ANOVA, Tukey's HSD).

FIGS. 6A, and 6B, show that oxaliplatin (Ox) treatment increases in caspase-1 activation reporter bioluminescence intensity in the hindpaw (A) and brain (B) as measured by area under the curve (AUC) were eliminated when the anti-epileptic seizure drug, lacosamide (Lac), was co-administered with Ox at 1 day and 3 days (n=5 mice per group; *p<0.05, **p<0.01, ***p<0.001, ****p<0.0001, repeated-ANOVA, Tukey's HSD).

FIG. 7 shows that lacosamide treatments attenuate Oxaliplatin (Ox)-induced changes in mouse grimace score. Mice were treated with either Ox or Ox+Lacosamide (Lac) every two days for 10 days (total 5 treatments). Statistically significant increase in MGS (i.e., more pain) at 24 h and 3 days after oxaliplatin treatment when compared with baseline in animals (n=5 mice per group; *p<0.05, two-way ANOVA, Tukey's HSD). Significant decrease in MGS scores (i.e., less pain) with 10 and 20 mg/kg oxaliplatin in combination with lacosamide treatments (*p<0.05, **p<0.01, ***p<0.001, ****p<0.0001, repeated-measures ANOVA, Tukey's HSD).

FIGS. 8A and 8B show cell viability effect of Oxaliplatin or Lacosamide as monotherapy and combination therapy in clinical colon cancer cell lines HCT-29 and HCT-116 cells. Cell viability was determined by measuring ATP content using CellTiter-Glo 2.0 reagent in HCT-29 (A) and HCT-116 (B) cells. The cells were grown in a monolayer format and treated with Oxaliplatin (Oxa), Lacosamide (Lac), or a combination of both. Treatments started at concentrations of 100 μM, followed by three-fold dilutions. Data represent mean±s.d., N=3 biological replicates, n=6 technical replicates.

### DETAILED DESCRIPTION

Disclosed herein are methods of reducing or inhibiting the development of chemotherapy induced peripheral neuropathy (CIPN) in a subject, pharmaceutical compositions comprising a platinum-based chemotherapy and a tetrodotoxin-dependent voltage gated sodium channel (NaV) inhibitor, and kits comprising a platinum-based chemotherapy and a tetrodotoxin-dependent voltage gated sodium channel (NaV) inhibitor.

### Methods of Reducing or Inhibiting the Development of Chemotherapy Induced Peripheral Neuropathy

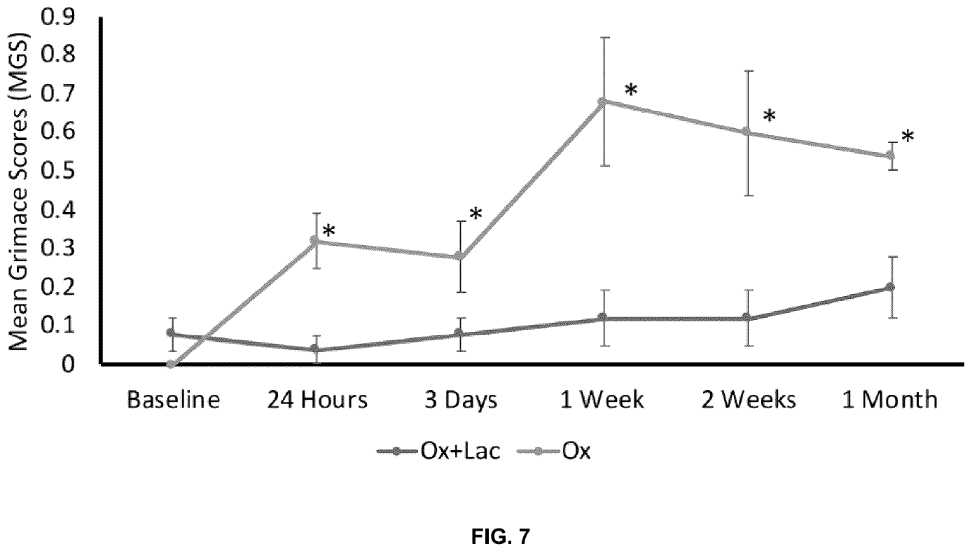

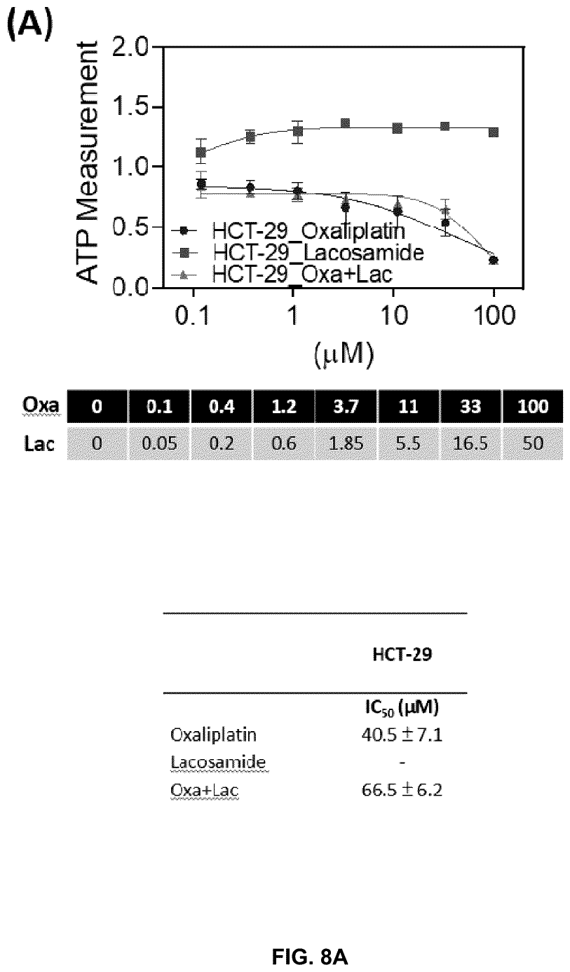

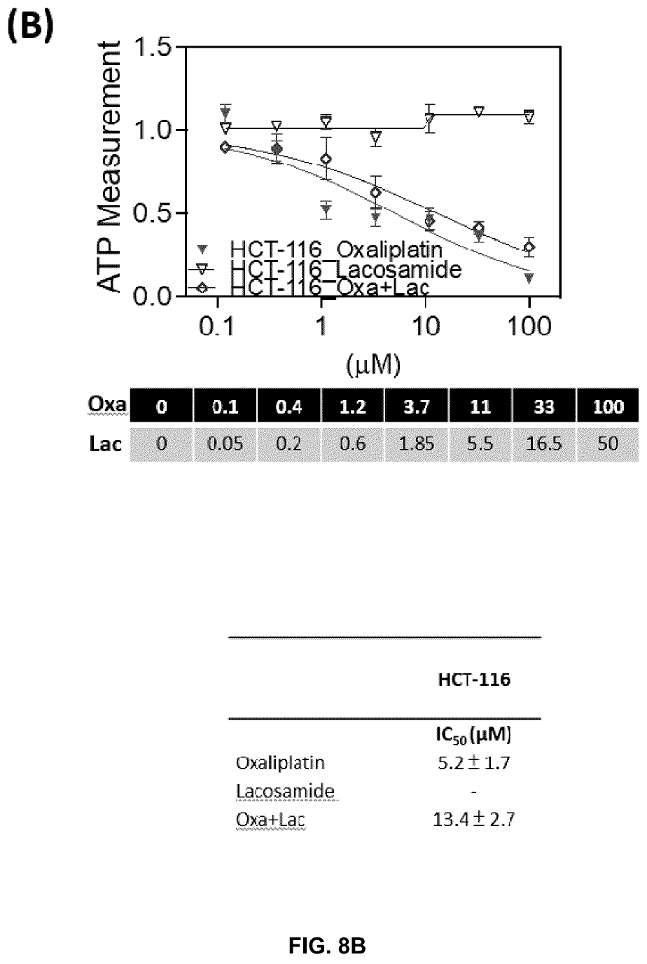

The inventors discovered that administration of the anti-seizure tetrodotoxin-dependent voltage gated sodium channel (NaV) inhibitor lacosamide in conjunction with the platinum-based chemotherapy oxaliplatin attenuated oxaliplatin-induced neuropathy in a mouse model (FIG. 7).

Accordingly, in an aspect of the current disclosure, methods of reducing or inhibiting the development of chemotherapy induced peripheral neuropathy (CIPN) in a subject in need thereof are provided. In some embodiments, the methods comprise administering a tetrodotoxin-dependent voltage gated sodium channel (NaV) inhibitor and a platinum-based chemotherapy to the subject.

Exemplary tetrodotoxin-dependent voltage gated sodium channel (NaV) inhibitors include, but are not limited to, lacosamide, suzetrigine, carbamazepine, and oxcarbmazepine. Additional drugs may include small molecule inhibitors QLS-81, Compound 100 and ST-2262, Sulfonamides such as PF-0508977 and GNE-616. In some embodiments, the NaV inhibitor is lacosamide.

As used herein, lacosamide refers to a composition having the structure:

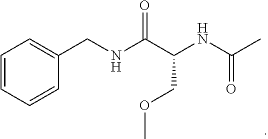 [SMILES: COC[C@@H](NC(C)=O)C(=O)NCc1ccccc1]

Suitable chemical derivatives or solvates of lacosamide may also be used in the disclosed methods, compositions, and kits.

Cancer treatments may necessitate higher or lower doses of lacosamide therapy depending on the oxaliplatin treatment paradigm.

There are a variety of “platinum-based chemotherapies” (used interchangeably herein with “platinum-based chemotherapeutics” and “platinum-based antineoplastics”) known in the art including, but not limited to, oxaliplatin, carboplatin, cisplatin, and nedaplatin. The methods may comprise administering a single platinum-based chemotherapy to a subject or may comprise administering at least one, or one or more platinum-based chemotherapy, e.g., 2, 3, 4, or more platinum-based chemotherapy to a subject in any combination.

The NaV inhibitor and the platinum-based chemotherapy may be administered in any order, e.g., the tetrodotoxin-dependent voltage gated sodium channel (NaV) inhibitor may be administered prior to the platinum-based chemotherapy, the tetrodotoxin-dependent voltage gated sodium channel (NaV) inhibitor may be administered at about the same time as the platinum-based chemotherapy, or the tetrodotoxin-dependent voltage gated sodium channel (NaV) inhibitor may be administered shortly after the platinum-based chemotherapy. In some embodiments, the NaV inhibitor is administered at about the same time as the platinum-based chemotherapy.

The NaV inhibitor and the platinum-based chemotherapy may be administered by any suitable route, e.g., intravenously, intraperitoneally, intrathecally, intramuscularly, orally, etc., at the discretion of a physician. The NaV inhibitor and the platinum-based chemotherapy may be administered by the same route, e.g., intravenously, or they may be administered by different routes, e.g., with the platinum-based chemotherapy being administered intravenously and the NaV being administered orally.

The methods may comprise administering a single NaV inhibitor or a combination of one or more NaV inhibitor.

As used herein, a subject in need thereof refers to a subject at risk of developing chemotherapy induced peripheral neuropathy (CIPN), e.g., a subject being administered a platinum-based chemotherapy, a subject that is in need of a platinum-based chemotherapy, or a subject that has already developed CIPN subsequent to being administered a platinum-based chemotherapy. A subject may be, e.g., a vertebrate, a mammal, e.g., a human, a dog, a cat, a horse, a mouse, a rat, or a non-human primate. In some embodiments, the subject is a human.

The inventors demonstrated that administration of oxaliplatin and lacosamide reduced mechanical allodynia and diminished grimace pain scores in mice as compared to mice administered only the platinum-based chemotherapy oxaliplatin. Accordingly, in some embodiments, the methods of this disclosure reduce mechanical allodynia and visual analog scale in the subject.

Platinum-based chemotherapies are typically administered to subjects that have been diagnosed with a cell proliferative disease or disorder, e.g., a cancer. Therefore, a subject in need thereof may comprise a subject that has been diagnosed with a cell proliferative disease or disorder, e.g., cancer. The cancer may comprise or consist of one or more of colorectal cancer, testicular cancer, ovarian cancer, lung cancer, bladder cancer, cervical cancer, breast cancer, head and neck cancer, endometrial cancer, prostate cancer, leukemia, sarcoma, stomach cancer, pancreatic cancer, esophageal cancer, uterine cancer, or brain cancer. A subject in need thereof may comprise a subject that is being administered a platinum-based chemotherapy for any purpose, as the inventor has demonstrated that addition of a NaV inhibitor, e.g., lacosamide, to a therapeutic regimen including platinum-based chemotherapeutics reduces CIPN in the subject.

The platinum-based chemotherapy and the NaV inhibitor may be administered with a further antineoplastic therapy in addition to the platinum-based chemotherapy and the NaV inhibitor, e.g., a chemotherapy, a radiation therapy, a hormone therapy, a targeted therapy, or an immunotherapy.

Provided herein are NaV inhibitors, e.g., lacosamide, for use in methods of treating CIPN in a subject or for use in a method of administering a platinum-based therapy to a subject, e.g., a platinum-based chemotherapy with reduced side effects.

Provided herein are NaV inhibitors, e.g., lacosamide, for use in the manufacture of a medicament for the treatment, attenuation, or prevention of CIPN.

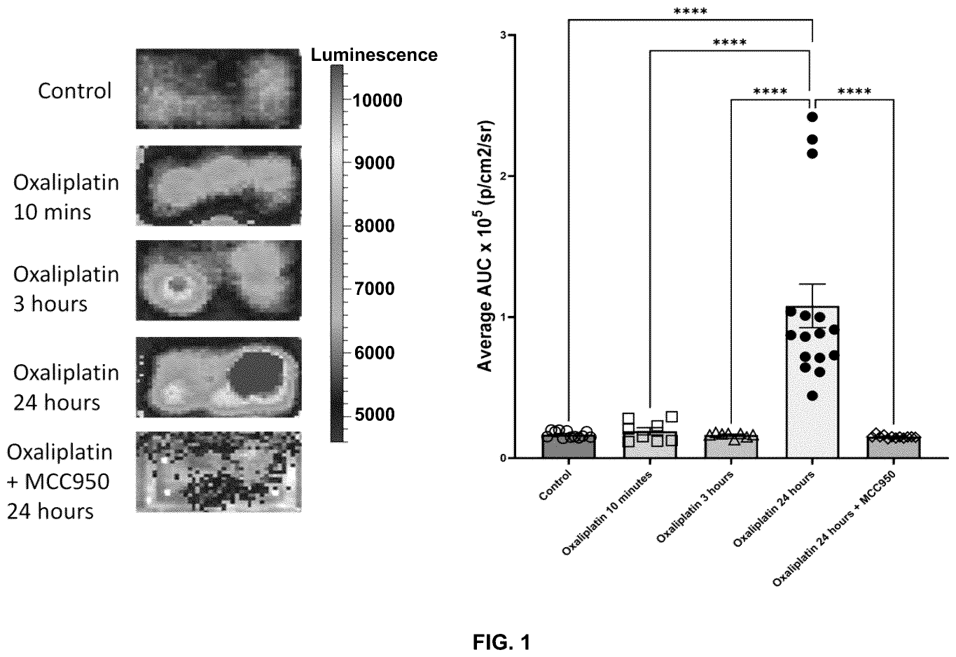

The inventors demonstrated that NLRP3 inhibitor MCC950 modulates sensory ganglia response to oxaliplatin and may directly influence pain states (FIG. 1).

Accordingly, disclosed herein are methods of treating chemotherapy induced peripheral neuropathy (CIPN) in a subject in need thereof. In some embodiments, the methods comprise administering a NOD, LRR, and pyrin domain-containing protein 3 (NLRP3) inhibitor to a subject that has been administered a platinum-based chemotherapy or a subject that is in need of a platinum-based chemotherapy.

An effective amount can be readily determined by the attending diagnostician, as one skilled in the art by the use of known techniques and by observing results obtained under analogous circumstances. In determining the effective amount or dose of compound, or compounds, administered, a number of factors can be considered by the attending diagnostician, such as: the species of the subject; its size, age, and general health; the degree of involvement or the severity of the disease or disorder involved; the response of the individual subject; the particular compound, or compounds, administered; the mode of administration; the bioavailability characteristics of the preparation administered; the dose regimen selected; the use of concomitant medication; and other relevant circumstances.

### Pharmaceutical Compositions

In an aspect of the current disclosure, pharmaceutical compositions are provided. In some embodiments, the pharmaceutical compositions comprise a NaV inhibitor and a platinum-based chemotherapy. The pharmaceutical composition may be provided as a formulation, e.g., for intravenous administration, or for any suitable route of administration as determined by a physician.

Pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-. 1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Kits

In an aspect of the current disclosure, kits are provided. In some embodiments, the kits comprise a platinum-based chemotherapy and a tetrodotoxin-dependent voltage gated sodium channel (NaV) inhibitor. The kits may further comprise instructions for using the kits for any of the disclosed methods.

The NaV inhibitor may be formulated for administration by any suitable route. The platinum-based chemotherapy may be formulated for administration by any suitable route.

### EMBODIMENTS

Embodiment 1) A method comprising administering a tetrodotoxin-dependent voltage gated sodium channel (NaV) inhibitor and a platinum-based chemotherapy to a subject.

Embodiment 2) A method of reducing or inhibiting the development of chemotherapy induced peripheral neuropathy (CIPN) in a subject in need thereof, the method comprising administering a tetrodotoxin-dependent voltage gated sodium channel (NaV) inhibitor and a platinum-based chemotherapy to the subject.

Embodiment 3) The method of any one of the preceding embodiments, wherein the tetrodotoxin-dependent voltage gated sodium channel (NaV) inhibitor is administered prior to the platinum-based chemotherapy, the tetrodotoxin-dependent voltage gated sodium channel (NaV) inhibitor is administered at about the same time as the platinum-based chemotherapy, or the tetrodotoxin-dependent voltage gated sodium channel (NaV) inhibitor is administered after the platinum-based chemotherapy.

Embodiment 4) The method of any one of the preceding embodiments, wherein the NaV inhibitor is administered at about the same time as the platinum-based chemotherapy.

Embodiment 5) The method of any one of the preceding embodiments, wherein the NaV inhibitor is lacosamide.

Embodiment 6) The method of any one of the preceding embodiments, wherein the platinum-based chemotherapy comprises oxaliplatin, carboplatin, cisplatin, or nedaplatin.

Embodiment 7) The method of any one of the preceding embodiments, wherein the platinum-based chemotherapy comprises oxaliplatin.

Embodiment 8) The method of any one of the preceding embodiments, wherein the subject is a mammal.

Embodiment 9) The method of any one of the preceding embodiments, wherein the subject is a human, a dog, a cat, a horse, a mouse, a rat, or a non-human primate.

Embodiment 10) The method of embodiment 9, wherein the subject is a human.

Embodiment 11) The method of any one of the preceding embodiments wherein the method reduces mechanical allodynia in the subject.

Embodiment 12) The method of any one of the preceding embodiments, wherein the subject is being administered a platinum-based chemotherapy for the treatment of a cell proliferative disease or disorder.

Embodiment 13) The method of embodiment 12, wherein the cell proliferative disease or disorder comprises cancer.

Embodiment 14) The method of embodiment 13, wherein the cancer comprises colorectal cancer, testicular cancer, ovarian cancer, lung cancer, bladder cancer, cervical cancer, breast cancer, head and neck cancer, endometrial cancer, prostate cancer, leukemia, sarcoma, stomach cancer, pancreatic cancer, esophageal cancer, uterine cancer, or brain cancer.

Embodiment 15) The method of embodiment 14, wherein the cancer comprises colorectal cancer.

Embodiment 16) The method of embodiment 14, wherein the cancer comprises breast cancer.

Embodiment 17) A method of treating chemotherapy induced peripheral neuropathy (CIPN) in a subject in need thereof, the method comprising administering a tetrodotoxin-dependent voltage gated sodium channel (NaV) inhibitor to a subject that has been administered a platinum-based chemotherapy.

Embodiment 18) The method of embodiment 17, wherein the NaV inhibitor is lacosamide.

Embodiment 19) The method of embodiment 17 or 18, wherein the platinum-based chemotherapy comprises oxaliplatin, carboplatin, cisplatin, or nedaplatin.

Embodiment 20) The method of any one of embodiments 17-19, wherein the platinum-based chemotherapy comprises oxaliplatin.

Embodiment 21) The method of any one of embodiments 17-20, wherein the subject is a mammal.

Embodiment 22) The method of any one of embodiments 17-21, wherein the subject is a human, a dog, a cat, a horse, a mouse, a rat, or a non-human primate.

Embodiment 23) The method of embodiment 22, wherein the subject is a human.

Embodiment 24) The method of any one of embodiments 17-23, wherein the method reduces mechanical allodynia in the subject.

Embodiment 25) The method of any one of embodiments 17-24, wherein the subject is being administered a platinum-based chemotherapy for the treatment of a cell proliferative disease or disorder.

Embodiment 26) The method of embodiment 25, wherein the cell proliferative disease or disorder comprises cancer.

Embodiment 27) The method of embodiment 26, wherein the cancer comprises colorectal cancer, testicular cancer, ovarian cancer, lung cancer, bladder cancer, cervical cancer, breast cancer, head and neck cancer, endometrial cancer, prostate cancer, leukemia, sarcoma, stomach cancer, pancreatic cancer, esophageal cancer, uterine cancer, or brain cancer.

Embodiment 28) The method of embodiment 27, wherein the cancer comprises colorectal cancer.

Embodiment 29) The method of embodiment 27, wherein the cancer comprises breast cancer.

Embodiment 30) A method of treating chemotherapy induced peripheral neuropathy (CIPN) in a subject in need thereof, the method comprising administering a NOD, LRR, and pyrin domain-containing protein 3 (NLRP3) inhibitor to a subject that has been administered a platinum-based chemotherapy.

Embodiment 31) The method of embodiment 30, wherein the NLRP3 inhibitor is administered prior to the platinum-based chemotherapy, the NLRP3 inhibitor is administered at about the same time as the platinum-based chemotherapy, or the NLRP3 inhibitor is administered after the platinum-based chemotherapy.

Embodiment 32) The method of embodiment 30 or 31, wherein the NLRP3 inhibitor is administered at about the same time as the platinum-based chemotherapy.

Embodiment 33) The method of any one of embodiments 30-32, wherein the NLRP3 inhibitor is MCC950, Usnoflast, or Verapamil.

Embodiment 34) The method of any one of embodiments 30-33, wherein the platinum-based chemotherapy comprises oxaliplatin, carboplatin, cisplatin, or nedaplatin.

Embodiment 35) The method of any one of embodiments 30-34, wherein the platinum-based chemotherapy comprises oxaliplatin.

Embodiment 36) The method of any one of embodiments 30-35, wherein the subject is a mammal.

Embodiment 37) The method of any one of embodiments 30-36, wherein the subject is a human, a dog, a cat, a horse, a mouse, a rat, or a non-human primate.

Embodiment 38) The method of embodiment 37, wherein the subject is a human.

Embodiment 39) The method of any one of embodiments 30-38, wherein the method reducing mechanical allodynia in the subject.

Embodiment 40) The method of any one of embodiments 30-39, wherein the subject is or has received a platinum-based chemotherapy for the treatment of a cell proliferative disease or disorder.

Embodiment 41) The method of embodiment 40, wherein the cell proliferative disease or disorder comprises cancer.

Embodiment 42) The method of embodiment 41, wherein the cancer comprises colorectal cancer, testicular cancer, ovarian cancer, lung cancer, bladder cancer, cervical cancer, breast cancer, head and neck cancer, endometrial cancer, prostate cancer, leukemia, sarcoma, stomach cancer, pancreatic cancer, esophageal cancer, uterine cancer, or brain cancer.

Embodiment 43) The method of embodiment 42, wherein the cancer comprises colorectal cancer.

Embodiment 44) The method of embodiment 42, wherein the cancer comprises breast cancer.

Embodiment 45) The method of any one of the preceding embodiments, the method further comprising administering an antineoplastic therapy to the subject in addition to the platinum-based chemotherapy.

Embodiment 46) The method of embodiment 45, wherein the antineoplastic therapy comprises a chemotherapy, a radiation therapy, a hormone therapy, a targeted therapy, or an immunotherapy.

Embodiment 47) A pharmaceutical composition comprising a platinum-based chemotherapy and a tetrodotoxin-dependent voltage gated sodium channel (NaV) inhibitor.

Embodiment 48) The pharmaceutical composition of embodiment 47, wherein the NaV inhibitor comprises lacosamide.

Embodiment 49) The pharmaceutical composition of embodiment 47 or 48, wherein the platinum-based chemotherapy comprises oxaliplatin, carboplatin, cisplatin, or nedaplatin.

Embodiment 50) The pharmaceutical composition of any one of embodiments 47-49, wherein the platinum-based chemotherapy comprises oxaliplatin.

Embodiment 51) A kit comprising a platinum-based chemotherapy and a tetrodotoxin-dependent voltage gated sodium channel (NaV) inhibitor.

Embodiment 52) The kit of embodiment 51, wherein the NaV inhibitor comprises lacosamide.

Embodiment 53) The kit of embodiment 51 or 52, wherein the platinum-based chemotherapy comprises oxaliplatin, carboplatin, cisplatin, or nedaplatin.

Embodiment 54) The kit of any one of embodiments 51-53, wherein the platinum-based chemotherapy comprises oxaliplatin.

### Further Definitions

The present invention is described herein using several definitions, as set forth below and throughout the application.

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### EXAMPLES

The following Examples are illustrative and should not be interpreted to limit the scope of the claimed subject matter.

### Example 1—Lacosamide Treatments Attenuate Oxaliplatin-Induced Neuropathy

TLR4-mediated inflammatory responses are known to play a critical role in a number of pain states, including chemotherapeutic treatment with oxaliplatin. Remarkably, the neuroinflammation which is associated with TLR4 is not limited to the peripheral nervous system (PNS) because it can also extend into the central nervous system (CNS) and potentially intensifies the processing of pain signals at the spinal level, thus enhancing overall nociceptive signaling. Taken together, evidence strongly suggests a persistent state of peripheral and central neuroinflammation that may contribute to amplified nociceptive signaling.

It is relatively well known that exposure to oxalip3latin induces a striking upregulation of inflammatory genes in dorsal root ganglia (DRG) and a release of proinflammatory cytokines and chemokines from macrophages, including IL1β, IL6, and TNFα^{1,2} However, the signaling pathways leading to cytokine release, as well as the contributions of these to the development of oxaliplatin-induced mechanical hyperalgesia, are not fully understood. The NLR family pyrin domain containing 3 (NLRP3) inflammasome, which is involved in IL1β release, is a strong candidate. Inflammasomes are multiprotein complexes which assemble and induce the activation of inflammatory cascades. Activation of the NLRP3 inflammasome and the production of IL-1β and IL-18 are tightly regulated and require two triggering steps, a priming step and an activation step utilizing the canonical caspase-1, an aspartyl protease, that promotes the cleavage of pro-inflammatory cytokine precursors, pro-IL-1β and pro-IL-18, to produce IL-1β and IL-18. IL-1β has been directly implicated in pain generation while increased IL-18 levels in both human sera and mouse brain have been associated with chronic pain states.³ More importantly, it has been suggested that oxaliplatin elicits the release of IL-18 and IL-1β in humans and mice and potentially drives oxaliplatin-induced painful neuropathy (OINP).⁴

To better understand the spatiotemporal kinetics of inflammatory processes associated with OINP, the inventors utilized ex vivo IVIS imaging of acutely dissociated dorsal root ganglion (DRG) tissue derived from a caspase-1 activation luciferase reporter mouse.⁵ Cultured DRGs derived from the transgenic mice were treated with oxaliplatin for 10 mins, 3 hours, and 24 hours. A final group was subjected to oxaliplatin and the NLRP3 inhibitor, MCC950 (FIG. 1). Significant increases in bioluminescence signals were observed in this nervous tissue treated with oxaliplatin at 24 hours. In contrast, the bioluminescence signal was nearly absent in culture wells subjected to oxaliplatin in combination with MCC950 (n=5 animals per group; 4 wells of 6-well rectangular plate per animals, ****p<0.0001, one way ANOVA, Tukey's HSD). This evidence suggests that NLRP3 inflammasomes can modulate sensory ganglia response to oxaliplatin and may directly influence pain states.

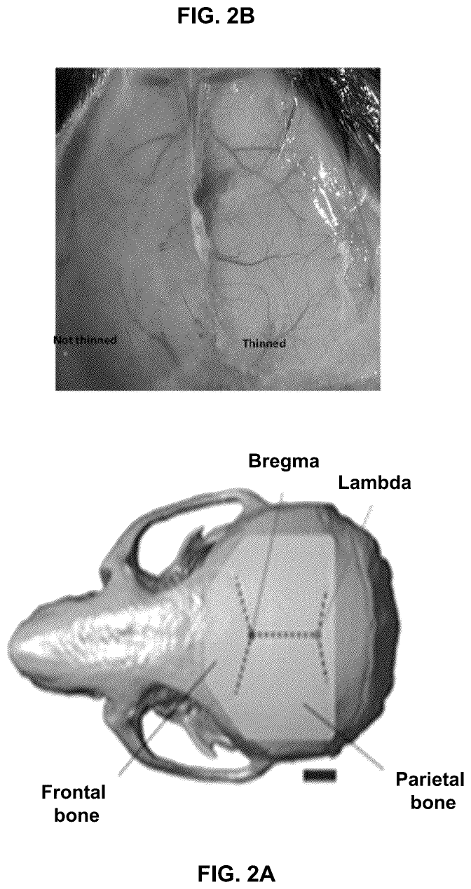

Next, the inventors used a caspase-1 activation luciferase reporter mouse model together with a thinned-skull cranial window preparation⁶⁻⁸ to disseminate inflammatory dynamics within the same animals over time in both the brain and hind paws. The inventors subjected mice to a thinned-skull cranial window procedure as described in the inventors' previous publications (FIG. 2).6-8 These thinned-skull cranial windows were minimally invasive and did not affect caspase-1 mediated inflammation in either male or female caspase-1 activation reporter mice following the short procedure. To demonstrate the lack of effect elicited by this preparation animals were imaged 2 weeks after cranial window thinning and the inventors found only a relatively low inflammatory signal. This result indicates that the window preparation has minimal effects in the caspase-1 associated inflammatory response.⁶

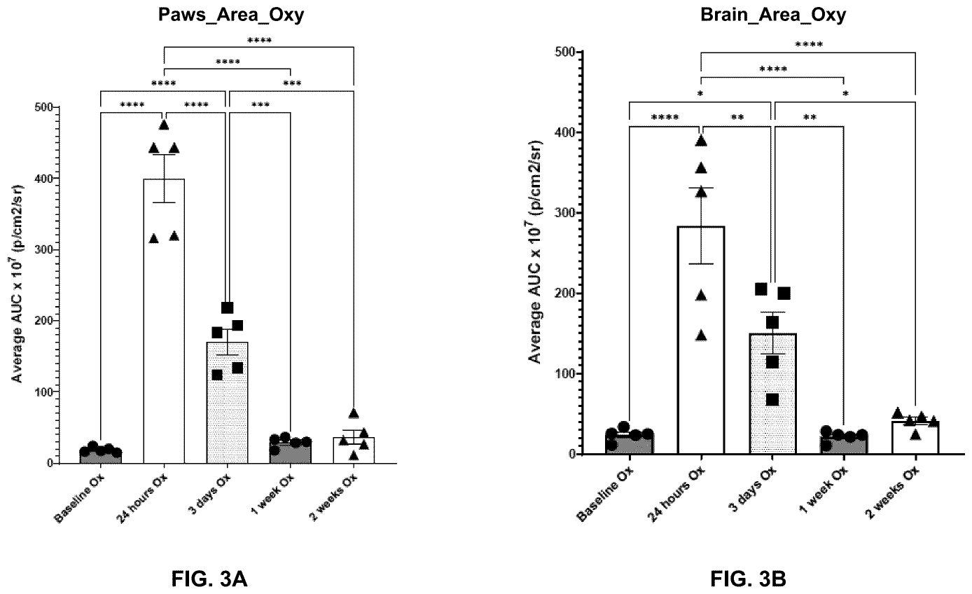

The long-term in vivo monitoring of caspase-1 activation-associated bioluminescence following the oxaliplatin treatment paradigm revealed an ongoing inflammation following cessation of the chemotherapeutic drug treatment in the brain region and hindpaws. Notably, significantly elevated brain and paw caspase-1 activation-associated signals in injured mice were detected at 24 hours post-treatment and sustained up to at least 3 days after the last injection of oxaliplatin (FIG. 3A & 3B).

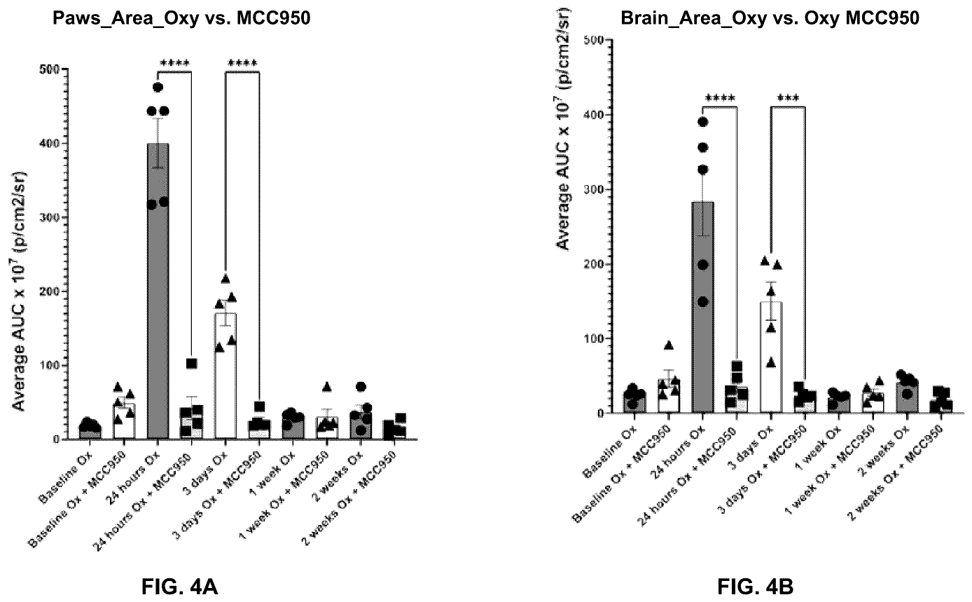

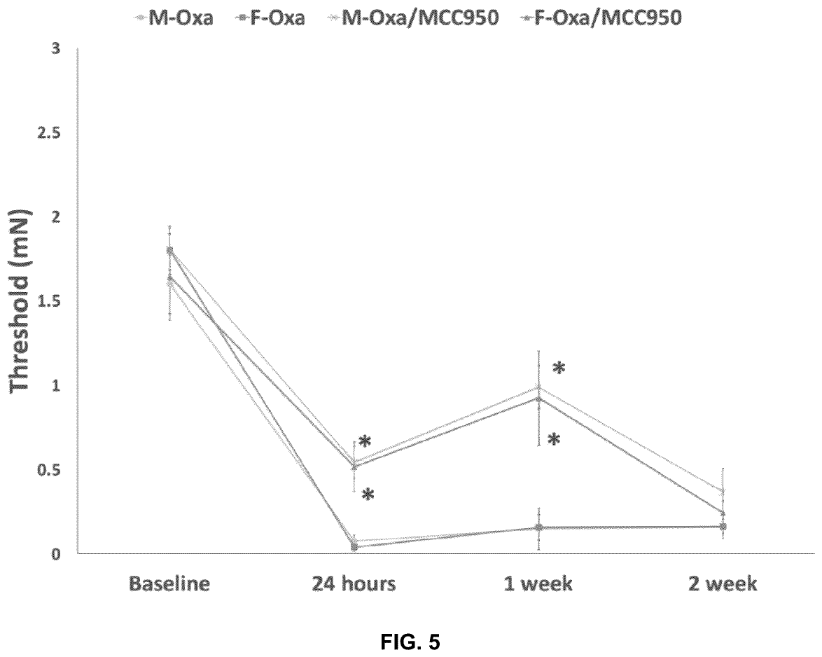

The inventors next determined whether the interruption of NLRP3 activation would influence oxaliplatin-induced inflammatory states. In these experiments, the inventors treated mice with MCC950 intraperitoneally shortly before each injection of oxaliplatin (total 6 treatments across injury timeline). Using vivo IVIS imaging, the inventors observed a significant reduction in bioluminescent signals at both 1 day and 3 days after cessation of the oxaliplatin treatment paradigm in both brain tissue and hind paws of MCC950 treatment groups compared to saline-treated groups (FIG. 4A, B). Given the ability of MCC950 to alter in vivo signaling in oxaliplatin-treated mice, the inventors then assayed the degree to which the NLRP3 inhibitor affected OIPN. Mice were treated with either 10 mg/kg oxaliplatin or 10 mg/kg oxaliplatin in combination with 5 mg/mL MCC950 intraperitoneally every two days for 10 days (total 5 treatments). Oxaliplatin-treated animals exhibited increased hypersensitivity of hind paw withdrawal to tactile stimulus in both male and female animals beginning at 24 hours and lasting for at least 2 weeks post-oxaliplatin treatment. In contrast, mice subjected to oxaliplatin in combination with MCC950 exhibited diminished mechanical pain hypersensitivity for at least 1 week (FIG. 5).

It has become evident from these experiments that the effect of MCC950 on oxaliplatin-induced painful neuropathy may be dependent on the NLRP3-caspase-1 activation signal as illuminated by in vivo cellular cascades and pain behavior in mice. Though largely unknown, there is compelling evidence that the elements upstream of NLRP3 signaling events include pattern recognition receptors (PRRs) which recognize endogenous DAMPs (Damage-associated molecular pattern molecules) and PAMPs (Pathogen-associated molecular pattern molecules). One such PRR which appears to be instrumental in driving OIPN is the toll-like receptor 4, TLR4.⁹ As TLR4 appears to play an important role in the ‘induction’ and ‘maintenance’ of CIPN in vivo⁹ and has been demonstrated to alter nociceptor excitability via tetrodotoxin-dependent voltage-gated sodium channels (NaVs),¹⁰ the inventors next attempted to elucidate the degree to which the first-in-class functionalized amino acid anti-seizure drug, lacosamide (LCM), influences caspase-1 activation following oxaliplatin treatment paradigm. This prediction is based on the inventors' previous work detailing the impact of LCM on sodium channel inactivation.¹¹ Although LCM shares a range of dose-dependent adverse effects with traditional anti-seizure drugs it has several advantages in that it does not induce hepatic drug metabolizing enzymes and has less risk of drug interactions and idiosyncratic adverse effects.

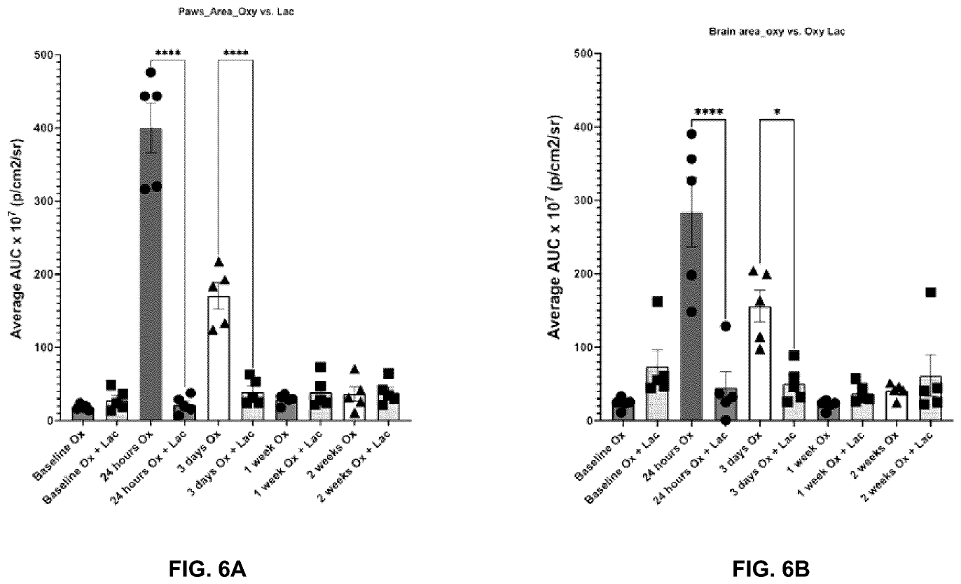

To test the ability of LCM to influence oxaliplatin-induced inflammatory states, the inventors treated mice with LCM intraperitoneally shortly before each injection of oxaliplatin (total 5 treatments across 10 days injury timeline). Using in vivo IVIS imaging, the inventors observed a significant reduction in bioluminescent signals not unlike MCC950 at both 1 day and 3 days after cessation of the oxaliplatin treatment paradigm in both brain tissue and hind paws of LCM treatment groups compared to saline-treated groups (FIG. 6A, 6B). Given the ability of LCM to alter in vivo signaling in oxaliplatin-treated mice, the inventors then assayed the degree to which the LCM affected OIPN as measured by mouse grimace scale (MGS). MGS offers a standardized behavioral coding system to study subjective pain experience in mice. Subsequently mice were treated with either 10 mg/kg oxaliplatin or 10 mg/kg oxaliplatin in combination with 5 mg/mL LCM intraperitoneally every two days for 10 days (total 5 treatments). Oxaliplatin-treated animals exhibited increased grimace scores in both male and female animals beginning at 24 hours and lasting for at least 1-month post-oxaliplatin treatment. In contrast, mice subjected to oxaliplatin in combination with LCM exhibited diminished grimace scores over that same period of time (FIG. 7).

Taken together, the inventors' study reveals a role for oxaliplatin-induced NLRP3 activation and development of OIPN. The inventors will proceed with control experiments utilizing known modulators of pain states including the seizure/neuropathic pain drug, gabapentin. Understanding the effects of anti-epileptic drugs on neuroinflammation states and cells within both the PNS and CNS constitutes a completely new approach to future treatment of OIPN.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A method of reducing or inhibiting the development of chemotherapy induced peripheral neuropathy (CIPN), or treating CIPN in a subject in need thereof, the method comprising administering a tetrodotoxin-dependent voltage gated sodium channel (NaV) inhibitor and a platinum-based chemotherapy to the subject.

2. The method of claim 1, wherein the tetrodotoxin-dependent voltage gated sodium channel (NaV) inhibitor is administered prior to the platinum-based chemotherapy, the tetrodotoxin-dependent voltage gated sodium channel (NaV) inhibitor is administered at about the same time as the platinum-based chemotherapy, or the tetrodotoxin-dependent voltage gated sodium channel (NaV) inhibitor is administered after the platinum-based chemotherapy.

3. The method of claim 1, wherein the NaV inhibitor is lacosamide.

4. The method of claim 1, wherein the platinum-based chemotherapy comprises oxaliplatin, carboplatin, cisplatin, or nedaplatin.

5. The method of claim 1, wherein the subject is a mammal.

6. The method of claim 1, wherein the subject is being administered a platinum-based chemotherapy for the treatment of a cell proliferative disease or disorder.

7. The method of claim 6, wherein the cell proliferative disease or disorder is a cancer.

8. The method of claim 7, wherein the cancer comprises colorectal cancer, testicular cancer, ovarian cancer, lung cancer, bladder cancer, cervical cancer, breast cancer, head and neck cancer, endometrial cancer, prostate cancer, leukemia, sarcoma, stomach cancer, pancreatic cancer, esophageal cancer, uterine cancer, or brain cancer.

9. A method of treating chemotherapy induced peripheral neuropathy (CIPN) in a subject in need thereof, the method comprising administering a NOD, LRR, and pyrin domain-containing protein 3 (NLRP3) inhibitor to a subject that has been administered a platinum-based chemotherapy or is in need of platinum-based chemotherapy.

10. The method of claim 9, wherein the NLRP3 inhibitor is administered prior to the platinum-based chemotherapy, the NLRP3 inhibitor is administered at about the same time as the platinum-based chemotherapy, or the NLRP3 inhibitor is administered after the platinum-based chemotherapy.

11. The method of claim 9, wherein the NLRP3 inhibitor is MCC950, Usnoflast, or Verapamil.

12. The method of claim 9, wherein the platinum-based chemotherapy comprises oxaliplatin, carboplatin, cisplatin, or nedaplatin.

13. The method of claim 9, wherein the subject is a mammal.

14. The method claim 9, wherein the subject is or has received a platinum-based chemotherapy for the treatment of a cell proliferative disease or disorder.

15. The method of claim 14, wherein the cell proliferative disease or disorder is a cancer.

16. The method of claim 15, wherein the cancer comprises colorectal cancer, testicular cancer, ovarian cancer, lung cancer, bladder cancer, cervical cancer, breast cancer, head and neck cancer, endometrial cancer, prostate cancer, leukemia, sarcoma, stomach cancer, pancreatic cancer, esophageal cancer, uterine cancer, or brain cancer.

17. The method of claim 9, the method further comprising administering an antineoplastic therapy to the subject in addition to the platinum-based chemotherapy.

18. A kit or pharmaceutical composition comprising a platinum-based chemotherapy and a tetrodotoxin-dependent voltage gated sodium channel (NaV) inhibitor.

19. The pharmaceutical composition or kit of claim 18, wherein the NaV inhibitor comprises lacosamide.

20. The pharmaceutical composition or kit of claim 19, wherein the platinum-based chemotherapy comprises oxaliplatin, carboplatin, cisplatin, or nedaplatin.
